(19) United States
(12) Patent Application Publication
(10) Pub. No.: US 2026/0224879 A1
(43) Pub. Date: Aug. 6, 2026

(54) RELEASABLE COUPLING DEVICE BETWEEN CATHETER AND COLLECTION DEVICE

(71) Applicant: Charles John FANARAS, Bow, NH (US)

(72) Inventor: Charles John FANARAS, Bow, NH (US)

(21) Appl. No.: 19/042,592

(22) Filed: Jan. 31, 2025

(51) Int. Cl. A61M 39/12; A61M 27/00; A61M 39/10; A61M 39/22

(52) U.S. Cl. CPC A61M 39/12; A61M 27/00; A61M 39/22; A61M 2039/1044; A61M 2202/0496; A61M 2205/581; A61M 2205/582

(57) ABSTRACT

An improved catheter system comprising a catheter having a discharge outlet connected to a first coupling device and a flexible tube connected to a second coupling device with a collection device. One of the first and the second coupling devices comprises a male sleeve and the other of the first and the second coupling devices comprising a female sleeve. The female sleeve is sized to receive and form a fluid tight seal with the male sleeve. The first coupling device carries a first connection mechanism while the second coupling device carries a mating second connection mechanism. The connection mechanism facilitates connection and disconnection of the first and the second coupling devices from one another by simultaneously disconnecting the first and the second portions of the connection mechanism and sliding the first and the second coupling devices relative to and out of engagement with one another.

### BACKGROUND OF THE INVENTION

### Field of the Invention

The present invention relates to an improved releasable coupling device, which facilitates a secure fluid tight releasable connection between a catheter and a collection device, such as a leg bag, a thigh bag or a waist bag, or some other sack, pouch, etc., in order to facilitate collection of urine and periodic emptying of the

collection device as well as rapid and secure reconnection of the releasable coupling device in order to collect additional urine in the collection device.

### Description of Related Prior Art

As is well known in the art—see FIG. 1—a leading end of a catheter 4 has a fluid inlet 6 while an opposed discharge end of the catheter supports a discharge outlet 8 which is generally a hollow elastic female conically-shaped coupling. The catheter 4 is typically inserted within the urinary track of a patient, in a conventional manner, so that the fluid inlet 6 of the catheter communicates with the bladder of the patient. Once the leading end of the catheter 4 is suitably positioned within the bladder, an inflatable balloon 12, supported adjacent the leading end of the catheter 4 is inflated, in a conventional manner, in order to retain the fluid inlet 6 of the catheter 4 within the bladder so that urine can commence flowing into the fluid inlet 6 and along the internal passageway 10 (not shown in detail) of the catheter 4 to the discharge outlet 8 of the catheter 4.

A collection device 18, such as a leg bag, a thigh bag or a waist bag, has an inlet which is generally permanently connected to a first end of a first flexible tube 16 while a second opposed end of the first flexible tube 16 supports a rigid hollow conically-shaped male coupling C which is generally permanently connected to the first flexible tube 16. The conical exterior surface of the conically-shaped male coupling C of the collection device may either be smooth or may have a plurality of barbed or annular rings or barbs formed therein. In either instance, the elastic female coupling of the discharge outlet 8 of the catheter 4 is designed to surround and elastically engage with and retain the conically-shaped male coupling C of the first flexible tube 16 and provide a fluid tight seal therewith.

One problem associated with such prior art connection is that the elastic female coupling of the catheter 4 tends to be not sufficiently resilient enough and thus the conically-shaped male coupling C, connected to the first flexible tube 16, may eventually, over time, become inadvertently disconnected or separated from the discharge outlet 8′ of the catheter 4. Such disconnection is generally not noticed by the patient until after an undesired spillage or a mess typically occurs. As a result of such disconnection, urine may now flow through the catheter 4 and out of the discharge outlet 8 onto the surrounding environment. Since nothing is connected to the discharge outlet, the urine exiting from the discharge outlet 8 may fall onto an article of clothing, bedding, flooring, etc., thereby causing an undesired mess which must be eventually cleaned up.

Another problem is that in the event that the elasticity of the elastic female coupling is sufficiently resilient so that the conically-shaped male coupling C, connected to the first flexible tube 16, does not become inadvertently disconnected or separated from the elastic female coupling of the discharge outlet 8 of the catheter 4, then, often times, it is extremely difficult for the patient, or the service personnel, to easily disconnect the conically-shaped male coupling C from the elastic female coupling 8 of the catheter 4 for servicing of the collection device 18. Such difficulty in separating the conically-shaped mating male and female connectors C, 8 from one another, during servicing of the collection device 18, is especially difficult for an elder individual who may be wearing the catheter/collection device system.

A further problem is that while the collection device 18 is being emptied by the user or service personnel (which typically takes at least a few minutes or so), the flow of urine, from the catheter 4 to the discharge outlet 8 thereof, remains unhindered or unobstructed and is thus able to can continue flowing. This, in turn, can also result in an undesired mess which requires cleanup and increases the overall time and effort required to service, e.g., empty, the collection device 18.

### SUMMARY OF THE INVENTION

Wherefore, it is an object of the present invention to overcome the above mentioned shortcomings and drawbacks associated with the prior art techniques and systems for servicing a catheter/collection device.

A main object of the present invention is to replace the prior art direct interface or connection, between the catheter and a connector of the flexible tube, with separate mating first and second releasable coupling devices, with one end of each of the first and second releasable coupling devices respectively permanently connected with one of the catheter and the flexible tube so as to ensure a secure, permanent and fluid tight seal/leakproof connection therewith while the oppose ends of each of the first and second releasable coupling devices ensure a secure but releasable fluid tight seal/leakproof connection with one another.

Another object of the present invention is to provide a connection system, which ensures an adequate fluid tight connection and seal between the mating first and second (male and female) coupling devices while, at the same time, rendering it relatively easy to disconnect and reconnect the mating first and second coupling devices with one another when servicing or emptying of the collection device is desired.

A further object of the present invention is to provide a connection system which achieves an adequate fluid tight seal, between the mating first and second (male and female) coupling devices, generally by pushing the mating male and female coupling devices toward one another into sealing engagement with one another.

A still further object of the present invention is to provide a positive signal or indicator, e.g., an audible tactile sound, to a patient or service personnel only once a fluid tight seal is achieved by between the mating first and second (male and female) coupling devices so that the patient, or the service personnel, reliably knows that the mating first and second coupling devices are properly connected with one another.

A still further object of the present invention is to provide a flow valve, adjacent the inlet or within first coupling device, in order to interrupt completely the flow of urine out of the catheter, during servicing of the collection device, until such time that the collection device is again properly connected with the catheter.

Yet another object of the present invention is to provide a flow valve, adjacent the discharge outlet of or within the second coupling device, in order to interrupt completely the flow of urine out of the discharge outlet of the second coupling device, during servicing of the collection device, until such time that the collection device is again properly connected with the catheter.

The present invention also relates to an improved catheter system comprising: a catheter having a discharge outlet being connected to an inlet of a first coupling device; a second end of a first flexible tube being connected to a collection device while a first end of the first flexible tube being connected to an outlet of a second coupling device; one of the first and the second coupling devices comprising a male cylindrical sleeve and the other of the first and the second coupling devices comprising a female cylindrical sleeve, and the female cylindrical sleeve being sized to receive and form a fluid tight seal with the male cylindrical sleeve; and the first coupling device carrying a first portion of a connection mechanism while the second coupling device carrying a mating second portion of the connection mechanism, and the connection mechanism facilitates connection and disconnection of the first and the second coupling devices from one another by simultaneous disconnecting the first and the second portions of the connection mechanism and sliding the first and the second coupling devices relative to and out of engagement with one another.

The present invention also relates to an improved catheter system comprising: a catheter having a discharge outlet being connected to a first coupling device; a collection device having a second end of a first flexible tube being connected to an inlet of the collection device while a first end of the first flexible tube being connected to an outlet of a second coupling device; one of the first and the second coupling devices comprising a male cylindrical sleeve and the other of the first and the second coupling devices comprising a female cylindrical sleeve, the female cylindrical sleeve being sized to receive and form a fluid tight seal with and around the male cylindrical sleeve, and one of the male and the female cylindrical sleeve supporting an O-ring for forming the fluid tight seal therebetween; the first coupling device carrying a first portion of a connection mechanism while the second coupling device carrying a mating second portion of the connection mechanism, and the connection mechanism facilitating releasable connection and disconnection of the first and the second coupling devices from one another by simultaneous disconnecting the first and the second portions of the connecting mechanism and sliding the first and the second coupling devices away from one another and out of engagement with one another; and when the first and the second portions of the connection mechanism are disconnect from one another, a valve of the first coupling device automatically interrupts the flow of urine out through the discharge outlet and past the first coupling device.

The present invention also relates to a method of providing an improved catheter system, the method comprising: connecting a first coupling device to a discharge outlet of a catheter; connecting a second end of a first flexible tube to an inlet of a collection device and connecting a first end of the first flexible tube to an outlet of a second coupling device; providing one of the first and the second coupling devices with a male cylindrical sleeve and providing the other of the first and the second coupling devices with a female cylindrical sleeve, sizing the female cylindrical sleeve to receive and form a fluid tight seal with and around the male cylindrical sleeve, and supporting an O-ring on one of the male and the female cylindrical sleeves for forming the fluid tight seal therebetween; carrying a first portion of a connection mechanism on the first coupling device and carrying a mating second portion of the connection mechanism on the second coupling device, and facilitating releasable connection and disconnection of the first and the second coupling devices from one another, via the connection mechanism, by simultaneous disconnecting the first and the second portions of the connecting mechanism from one another and sliding the first and the second coupling devices away from one another and out of engagement with one another; and automatically interrupting, via a pair of movable valves, the flow of fluid through the first and the second coupling devices when the first and the second portions of the connection mechanism are disconnect from one another.

The present invention also relates to a kit for modifying a releasable connection system of a catheter system, the kit comprising: a first coupling device having an inlet for connection with a catheter; a second coupling device having an outlet for connection with a flexible tube connected with a collection device; one of the first and the second coupling devices comprising a male cylindrical sleeve and the other of the first and the second coupling devices comprising a female cylindrical sleeve, and the female cylindrical sleeve being sized to receive and form a fluid tight seal with the male cylindrical sleeve; and the first coupling device carrying a first portion of a quick connection mechanism while the second coupling device carrying a mating second portion of the quick connection mechanism, and the quick connection mechanism facilitates connection and disconnection of the first and the second coupling devices from one another by simultaneous disconnecting the first and the second portions of the quick connection mechanism and sliding the first and the second coupling devices relative to and out of engagement with one another; and when the first and the second coupling devices are disconnected from one another, a flow through at least one of the first and the second coupling devices is automatically interrupted.

### BRIEF DESCRIPTION OF THE DRAWINGS

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIG. 1 is a diagrammatic view showing a conventional catheter system according to the prior art;

FIG. 2 is a diagrammatic view, similar to FIG. 1, showing a first embodiment of the improved catheter system according to the disclosure;

FIG. 2A is an enlarged cross section of area 2A of FIG. 2;

FIG. 2B is a diagrammatic front elevational view of the locking collar of FIG. 2;

FIG. 3 is a diagrammatic view of the first and second coupling devices showing a second embodiment of the improved catheter system according to the disclosure;

FIG. 4 is a diagrammatic view of the first and second coupling devices showing a third embodiment of the improved catheter system according to the disclosure;

FIG. 5A is an enlarged cross section, similar to area 2A of FIG. 2, showing a fourth embodiment of the improved catheter system according to the disclosure;

FIG. 5B is a diagrammatic front elevational view of the locking collar of FIG. 5; and

FIG. 6 is a cross section showing a fifth embodiment of the improved catheter system according to the disclosure.

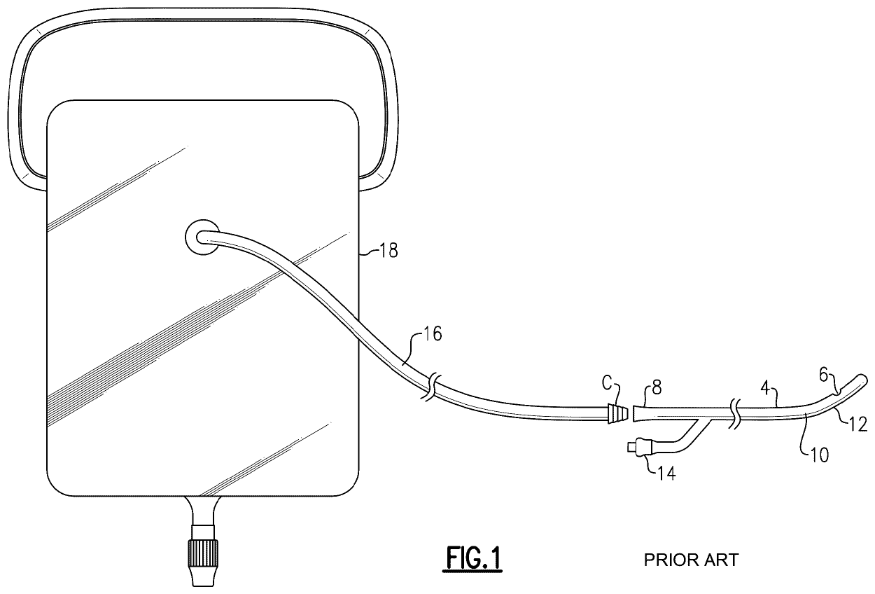

It should be understood that the drawings are not necessarily to scale and that the disclosed embodiments are sometimes illustrated diagrammatical and in partial views. In certain instances, details which are not necessary for an understanding of this disclosure or which render other details difficult to perceive may have been omitted. It should be understood, of course, that this disclosure is not limited to the particular embodiments illustrated herein.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention.

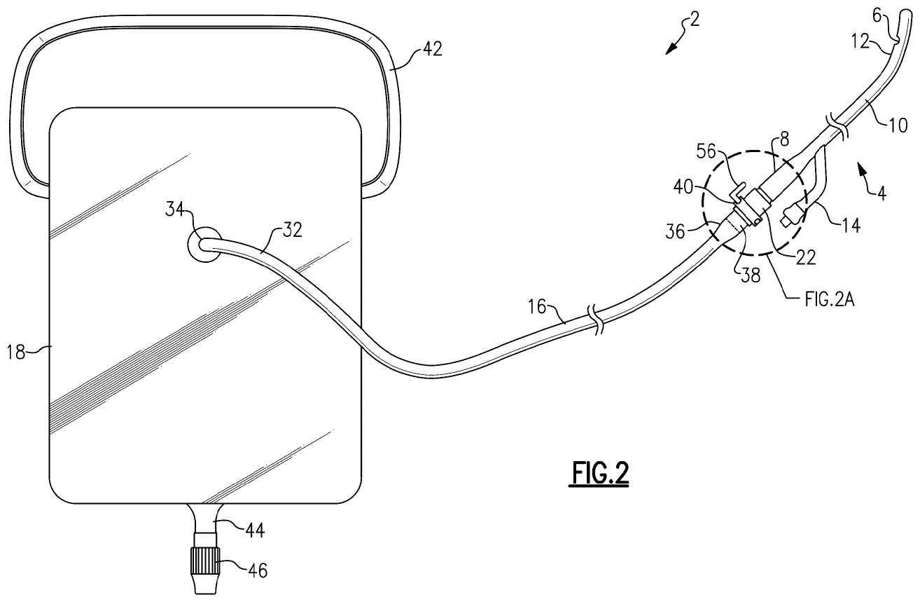

Turning now to FIG. 2, a brief description concerning the various components of a first embodiment of the present disclosure will now be briefly discussed.

As can be seen in this first embodiment, the improved catheter system 2 generally comprises a conventional catheter 4, which is typically installed within the urinary track of a patient so that the leading end thereof communicates with and is located within the bladder of the patient. A fluid inlet 6 is formed, adjacent the leading end of the catheter 4, and the fluid inlet 6 communicates with a (female conically shaped) discharge outlet 8 via an internal catheter passageway 10 (not shown in detail) which extends along the length of the catheter 4 from the fluid inlet 6 to the discharge outlet 8. As is conventional in the art, an inflatable balloon 12 (not shown in detail) is located adjacent the leading end of the catheter 4. Once the catheter 4 is properly positioned within the bladder, the balloon 12 is inflated, in a conventional manner via an inflation port 14 of the catheter 4, so as to prevent the inadvertent removal of the fluid inlet 6 of the catheter 4 from the bladder. As is conventional in the art, the catheter 4 is designed to permit a substantially unhindered flow of urine from the bladder, past the prostate, to the discharge outlet 8, along the first flexible tube 16 and eventually into a collection device 18 in order to remove urine from the bladder of the individual or patient in a substantially continuous manner.

As shown in FIG. 2A, a first (barbed) end 20 of a first coupling device 22 matingly engages with the female discharge outlet 8 of the catheter 4. It is important that a permanent fluid tight connection is achieved between the first (barbed) end 20 of the first coupling device 22 and the discharge outlet 8. For example, the first end 20 of a first coupling device 22 may be larger in size than the conventional coupling according to the prior art. That is, the first end 20 of the first coupling device 22 has a diameter which is at least 10% larger, more preferably at least 25% larger and most preferably at least 35% larger than the diameter of the discharge outlet 8 of the catheter 4. Such larger diameter of the first (barbed) end 20 of the first coupling device 22 ensures that the conically-shaped elastic female discharge outlet 8 becomes sufficiently stretched, when connected to the first (barbed) end 20 of the first coupling device 22, so that, for the entire life of the catheter 4, e.g., typically a time span of about 2-4 weeks or so, the first coupling device 22 will always remain permanently attached or affixed to the discharge outlet 8 of the catheter 4. Such connection ensures that no leakage will occur at the interface between those two devices. Alternatively and/or in addition thereto, the first end 20 of the first coupling device 22 may be permanently attached, affixed, or connected, e.g., shrunk fit, glued, adhered, bonded, melted, welded, etc., to the discharge outlet 8 of the catheter 4. In addition, it is possible that the female conically-shaped elastic discharge outlet 8 of the catheter 4 may be eliminated and the first end 20 of the first coupling device 22 may be directly connected with the internal catheter passageway 10 of the catheter 4 so that the first coupling device 22 thereby forms the discharge outlet 8 of the catheter 4. It is also possible that a second flexible tube (not shown) may interconnect or couple, in a fluid tight seal/leakproof manner, the first end 20 of the first coupling device 22 with the discharge outlet 8 of the catheter 4, without departing from the spirit and scope of the present disclosure.

A second end 24 of the first coupling device 22 includes a hollow female (or possibly a male) cylindrical sleeve 26 having a length of between about ¼ inch to about ⅝ inch or so and an inner diameter of about ½±¼ inch. A first coupling passageway 28 extends from the first end 20 of the first coupling device 22, through the female (or possibly a male) cylindrical sleeve 26, to the second end 24 of the first coupling device 22 to permit the flow of fluid, e.g., urine, through the first coupling device 22.

As shown in FIG. 2, a first end 32 of a hollow first flexible tube 16 is generally permanently connected, in a conventional manner, to fluid inlet 34 of the collection device 18, such as a leg bag, a thigh bag or a waist bag, while an opposed second end 36 of the first flexible tube 16 is permanently attached, affixed or connected, e.g., shrunk fit, glued, adhered, bonded, melted, welded, etc., to a second (outlet) end 38 of a second coupling device 40. If desired, a one way valve (not shown) may be provided either along a section of the first flexible tube 16 or adjacent the fluid inlet 34 of the collection device 18 to ensured that fluid only flows in the direction toward the collection device 18 and not inadvertently out of the collection device 18, via the first flexible tube 16, toward the second coupling device 40. As is conventional in the art, the collection device 18 is normally provided with a strap 42 for releasably securing the collection device 18 to the waist, the thigh, or the leg of a patient or some other component, in a conventional manner. A drainage port 44 facilitates periodic emptying of the collection device 18 once the collection device 18 becomes sufficiently full. This drainage port 44 typically includes a (rotatable) drainage valve 46 which has an “open” position (e.g., when rotated counter clockwise for example) to facilitate emptying of the collection device 18, when desired or necessary, and a “closed” position (e.g., when rotated clockwise for example) to facilitate collection of urine within the collection device 18. As such drainage feature is conventional and well known in the art, a further detail discussion concerning the same is not provided at this time.

As shown in FIG. 2A, a first end 48 of the second coupling device 40 includes a hollow male (or possibly a female) cylindrical sleeve 50 having a length of between about ¼ inch to about ½ inch or so and an outer diameter of about ½±¼ inch. A second coupling passageway 52 extends from the first end 48 of the second coupling device 40, through the male (or possibly a female) cylindrical sleeve 50, to the outlet 51 of the second coupling device 40 in order to permit the flow of fluid, e.g., urine, through the second coupling device 40 to the first flexible tube 16 and the collection device 18.

FIG. 2A specifically shows that second end 24 of the first coupling device 22, includes a female cylindrical sleeve 26 while the first end 48 of the second coupling device 40 includes a male cylindrical sleeve 50. It is to be appreciated, however, that the positions of the male and the female cylindrical sleeves 26, 50 may be switched or reversed, namely, the second end 24 of the first coupling device 22 may support the male cylindrical sleeve 50 while the first end 48 of the second coupling device 40 may support the female cylindrical sleeve 26 (this configuration is not shown), without departing from the spirit and scope of this disclosure.

The female cylindrical sleeve 26 is sized and shaped so as to closely surround and enclose the male cylindrical sleeve 50 and thereby form a fluid tight connection and seal between those two components with only a small clearance, e.g., 0.050+0.045 of an inch, being provided therebetween. As shown, at least one O-ring 53 is provided between the mating male and the female cylindrical sleeves 26, 50 so as to provide the fluid tight seal which prevents the flow of fluid therebetween. As shown, an annular channel or recess 54 is formed within the exterior surface of the male cylindrical sleeve 50, adjacent a leading end thereof, and the O-ring 53 is captively retained within the annular recess 54 and engages with the inwardly facing cylindrical surface of the female cylindrical sleeve 26 to form a fluid tight seal therewith. It is to be appreciated that the location of the O-ring 53 may be switched or reversed, namely, the annular channel or recess 54 may be formed in the interior surface of the female cylindrical sleeve 26 and the O-ring 53 may be captively retained within this annular recess 54 and engage with the outwardly facing surface of the male cylindrical sleeve 50 (this embodiment is not shown) to form a fluid tight seal therebetween, without departing from the spirit and scope of this disclosure.

When the female cylindrical sleeve 26 of the first coupling device 22 and the male cylindrical sleeve 50 of the second coupling device 40 are moved relative to one another in a direction toward and into engagement with one another, the male cylindrical sleeve 50 is at least partially received by and within the female cylindrical sleeve 26 with the O-ring 53 being sandwiched between the inwardly and the outwardly facing surfaces and forming a fluid tight seal between those two surfaces. That is, the male cylindrical sleeve 50 of the second coupling device 40 is at least partially surrounded and enclosed by the hollow female cylindrical sleeve 26 with the O-ring 53 located therebetween to achieve the fluid tight seal between those two components.

Thereafter, if the first coupling device 22, supporting the female cylindrical sleeve 26, and the second coupling device 40, supporting the male cylindrical sleeve 50, are moved relative to one another in a direction away from and out of engagement with one another, the male cylindrical sleeve 50 eventually becomes removed, disconnected and separated from the female cylindrical sleeve 26 so that the fluid tight seal, formed by the O-ring 53 located therebetween, is broken and the first coupling device 22 is separated and disconnected from the second coupling device 40. Thereafter, the collection device 18 may be serviced by the patient or service personnel, e.g., the urine may be emptied in a toilet or some other suitable receptacle in a conventional manner by actuation of the drainage valve 46.

To ensure that the first coupling device 22 and the second coupling device 40 reliably remain in engagement with one another during collection of fluid, the first coupling device 22 supports a first portion 56, e.g., a locking collar, of a mechanical quick connection mechanism 58 while the second coupling device 40 supports a mating second portion 60, e.g., an annular recess or groove, of the mechanical quick connection mechanism 58. As shown in FIGS. 2A and 2B, the first portion of the mechanical quick connection mechanism 58 comprises a locking collar 56 having a generally oval shaped opening 62 while the second portion of the mechanical quick connection mechanism 58 comprises an annular recess or groove 60 formed in the exterior surface of the male cylindrical sleeve 50 at a location spaced away from the O-ring 53 toward a downstream trailing end of the male cylindrical sleeve 50. A width of an upper U-shaped portion 61 of the oval shaped opening 62 is slightly larger than the outer diameter of the male cylindrical sleeve 50 while a radius of a lower semicircular shaped portion 63 of the oval shaped opening 62 is slightly larger than the outer radius of the annular groove 60 but also slightly smaller than the outer diameter of the male cylindrical sleeve 50 so that the lower semicircular shaped

portion 63 of the oval shaped opening 62 can be received and retained within the annular recess or groove 60 and thereby prevent lateral motion of the first coupling device 22 and the second coupling device 40 relative to one another. The locking portion 56 is accommodated within and slidable along a slot or channel 65 which extends normal to the cylindrical passageway 28 extending through the female cylindrical sleeve 26 adjacent the outlet of the first coupling device 22. Each opposed lateral side of the locking portion 56 has an outwardly extending small protrusion (not shown in detail) which passes over a mating inwardly extending small protrusion (not shown in detail) formed on the opposed lateral walls of the slot or channel 65 which permits limited sliding movement of the locking portion 56, within the slot or channel 65, while preventing inadvertent removal of the locking portion 56 from the slot or channel 65.

When engagement of the first coupling device 22 and the second coupling device 40 is desired, the locking collar 56 is biased by the patient or service personnel, against the action of the spring force (coil spring 59, or possibly a leaf spring), into a position in which the oval shaped opening 62 of the locking collar 56 is generally concentric with the cylindrical passageway 28 extending through the female cylindrical sleeve 26, i.e., the lower semicircular shaped portion of the oval shaped opening 62 of the locking collar 56 is forced toward the closed end 70 of the slot or channel 65 and thus does not generally obstruct the cylindrical passageway 28. When the locking collar 56 is in this position, the lower semicircular shaped portion of the oval shaped opening 62 is sufficiently spaced from the exterior surface of the male cylindrical sleeve 50 so that the exterior surface of the male cylindrical sleeve 50 can slide past the semicircular shaped portion 63, of the oval shaped opening 62, until the semicircular shaped portion 63, of the oval shaped opening 62, eventually reaches and is aligned with and can lockingly engage with the annular recess or groove 60 of the male cylindrical sleeve 50.

Once this occurs, the spring 59 automatically biases the locking collar 56 away from the close end of the slot or channel 65 into locking engagement with the annular recess or groove 60 so as to prevent any further lateral displacement or movement of the first coupling device 22 relative to the second coupling device 40. As soon as the locking collar 56 engages with the annular groove 60, a tactile sound is generated and this tactile sound may be heard and/or felt by the individual, e.g., the patient or the service personnel, achieving the locking engagement between the first and the second coupling devices 22, 40.

In the event that the locking collar 56 is again biased, against the action of the spring, into a position in which the oval shaped opening 62 of the collar is again concentric with the cylindrical passageway 28 extending through the cylindrical female sleeve 26, then the semicircular shaped portion 63 of the locking collar 56 becomes spaced and disengaged from the annular recess or groove 60 so that an operator, e.g., a patient or service personnel, may move the first and the second coupling devices 22, 40 laterally, relative to one another, to disengage and separate the first and the second coupling devices 22, 40 from one another.

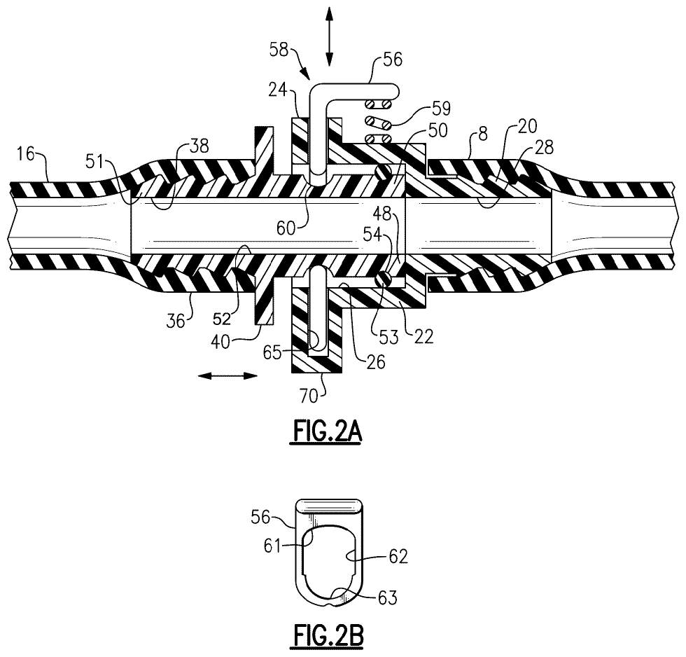

Turning now to FIG. 3, a brief description concerning the various components of a second embodiment of the present disclosure will now be briefly discussed in detail. As this embodiment is similar to the first embodiment in many respects, only the differences between the second embodiment and the first embodiment will be discussed. Similar or like elements in this embodiment are given the same reference numerals.

As can be seen in this Figure, the major difference between the first embodiment and the second embodiment, is the addition of a conventional first rotatable valve 64 at a location between the discharge outlet 8 of the catheter 4 and the first coupling device 22, e.g., preferably being part of or located closely adjacent the inlet of the first coupling device 22. The first rotatable valve 64 has a closed position, which prevents the flow of fluid, e.g., urine, and an open position, which permits the flow of fluid, e.g., urine. The first rotatable valve 64 is normally located in the open position in which fluid is permitted to flow from the catheter 4 to the collection device 18. When servicing of the collection device 18 is desired or required, the first rotatable valve 64 is moved into its closed position which prevents the flow of fluid, e.g., urine, past the first rotatable valve. Thereafter, the first and the second coupling devices 22, 40 may be disconnected from one another, as described above, and the collection device 18 may be removed and serviced. Once servicing of the collection device 18 is completed, the first and second coupling devices 22, 40 are again reconnected with one another and then the first rotatable valve 64 is moved, into its open position, to again permit the flow of fluid, e.g., urine, past the first rotatable valve 64 to the collection device 18.

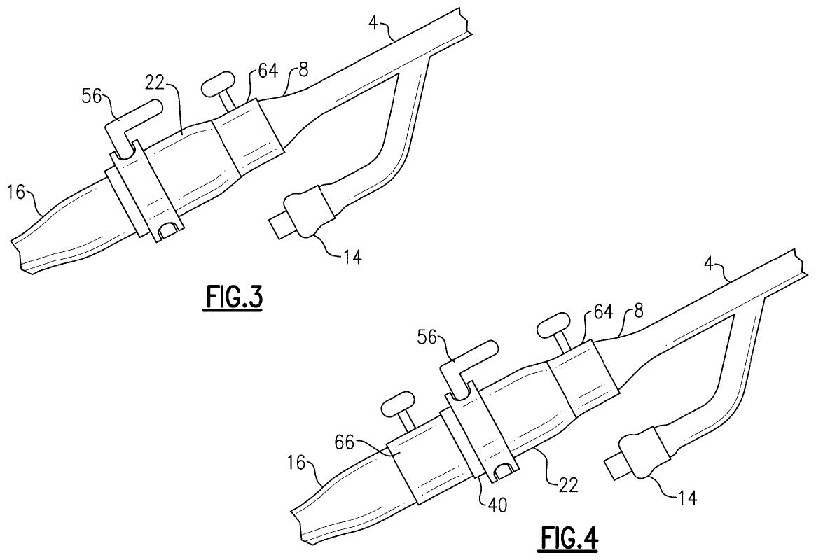

Turning now to FIG. 4, a brief description concerning the various components of a third embodiment of the present disclosure will now be briefly discussed in detail. As this embodiment is similar to the second embodiment in many respects, only the differences between the third embodiment and the first and the second embodiments will be discussed. Similar or like elements in this embodiment are given the same reference numerals.

As can be seen in this Figure, the major difference between the third embodiment and the second embodiment, is the addition of a second rotatable valve 66 at a location between the second coupling device 40 and the collection device 18, e.g., preferably being part of or located closely adjacent the outlet of the second coupling device 40. The second rotatable valve 66 has a closed position, which prevents the flow of fluid, e.g., urine, and an open position, which permits the flow of fluid, e.g., urine. The second rotatable valve 66 is normally located in the open position which permits the flow of urine from the second coupling device 40 to the collection device 18. When servicing of the collection device 18 is desired or required, the second rotatable valve 66 is moved into its closed position to prevent the flow of fluid, e.g., urine, past the second rotatable valve 66. The first rotatable valve 64 is also rotated into is closed position. Thereafter, the first and the second coupling devices 22, 40 may be disconnected from one another, as described above, and the collection device 18 may be removed and serviced. Once servicing of the collection device 18 is completed, the first and second coupling devices 22, 40 are again reconnected with one another and the first and the second rotatable valves 64, 66 are both then moved into their open positions to again permit the flow of fluid, e.g., urine, past the first and the second rotatable valves 64, 66 to the collection device 18.

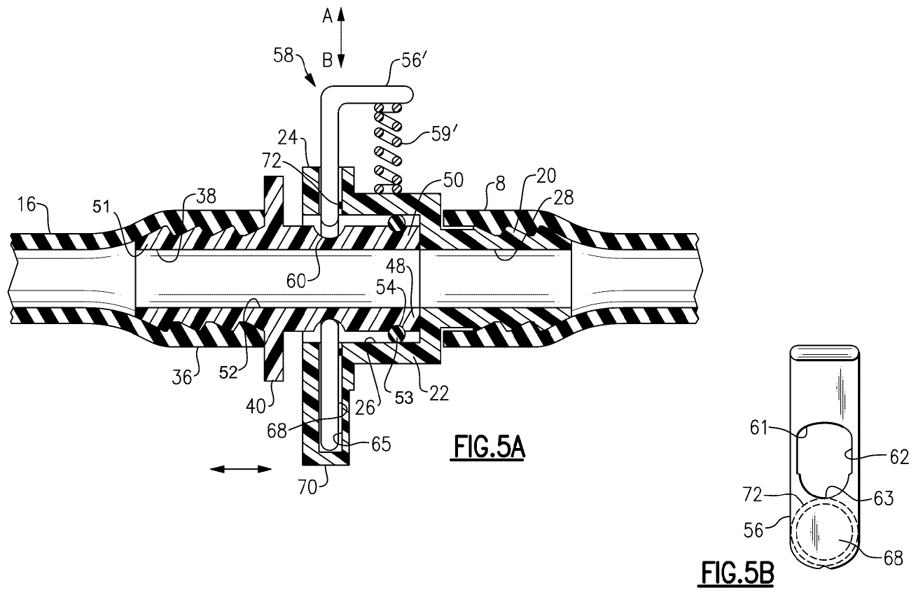

Turning now to FIG. 5A, a brief description concerning the various components of a fourth embodiment of the present disclosure will now be briefly discussed in detail. As this embodiment is similar to the first embodiment in many respects, only the differences between the fourth embodiment and the first embodiment will be discussed. Similar or like elements in this embodiment are given the same reference numerals.

As can be seen in this Figure, the major difference between the fourth embodiment and the first embodiment, is an increase in a length of the locking collar 56′ (see FIG. 5B) and an increase in length of the (compression) spring 59′. As a result, the locking collar 56′ has a much larger range of motion between its locking position and it sealing position. The spring 59′ normally biases the locking collar 56′ in the direction of arrow A into a closed sealing position where a lower rear surface 68 of the locking collar 56′ abuts against and seals the outlet opening of the first coupling passageway 28 so as to prevent the flow of fluid, e.g., urine, out of the first coupling passageway 28 and past the locking collar 56′. In this normally closed position, the rear surface 68 of locking collar 56′ completely covers the entire opening of the outlet of the first coupling passageway 28. If desired, the front face of the outlet of the first coupling device 22 (or the rear surface 68 of the locking collar 56′) may carry an O-ring 72, or some other conventional sealing member, to improve the fluid tight seal achieved between the rear surface 68 of the locking collar 56′ and the cylindrical outlet of the first coupling passageway 28. As a result, the locking collar 56′, when in its closed position, automatically prevents the flow of fluid through the outlet of the first coupling device 22 any time that the first coupling device 22 is disconnected from the second coupling device 40. That is, the spring 59′ biases the locking collar 56′ in the direction of arrow A as soon as the first and second coupling devices 22, 40 become disconnected from one another so that rear surface 68 completely covers the outlet of the first coupling passageway 28 so as to prevent the flow for fluid.

However, when connection of the first coupling device 22 with the second coupling device 40 is desired, the locking collar 56′, of the mechanical quick connection mechanism 58, is sufficiently depressed (in the direction of arrow B toward the closed end 70 of the slot or channel 65 to compress the spring 59′) into a position in which the oval shaped opening 62 of the locking collar 56 is generally concentric with the cylindrical passageway 28 extending through the female cylindrical sleeve 26, i.e., neither the lower semicircular shaped portion 63 of the oval shaped opening 62 of the locking collar 56 nor the rear surface 68 obstructs the cylindrical passageway 28. When the locking collar 56 is in this position, the lower semicircular shaped portion 63 of the oval shaped opening 62 is sufficiently spaced from the exterior surface of the male cylindrical sleeve 50 so that the exterior surface of the male cylindrical sleeve 50 can slide through the oval shaped opening 62, past the semicircular shaped portion 63, until the semicircular shaped portion 63 reaches and is aligned with the annular recess or groove 60 of the male cylindrical sleeve 50.

Once this occurs, the spring 59′ automatically biases the locking collar 56′ a small distance in the direction of arrow A into locking engagement with the annular recess or groove 60 so as to prevent any further lateral displacement or movement of the first coupling device 22 relative to the second coupling device 40. As soon as the locking collar 56′ engages with the annular recess or groove 60, a tactile sound is generated and this tactile sound or indication may be heard and/or felt by the individual, e.g., the patient or the service personnel, achieving the locking engagement between the first and the second coupling devices 22, 40.

In the event that the locking collar 56′ is again biased, initially in the direction of arrow B a small distance against the action of the spring 59′, into a position in which the oval shaped opening 63 of the locking collar 56′ is again concentric with the cylindrical passageway 28 extending through the cylindrical female sleeve 26, then the locking collar 56′ becomes spaced and disengaged from the annular recess or groove 60 so that an operator, the patient or service personnel, may move the first and the second coupling devices 22, 40 laterally, relative to one another, to disengage and separate the first and the second coupling devices 22, 40 from one another. As soon as this disengagement occurs, the spring 59′ thereafter automatically biases the locking collar 56′, in the direction of arrow A, into its closed position in which the lower rear surface 68 of the locking collar 56′ abuts against and completely covers and seals the outlet opening of the first coupling passageway 28 so as to prevent the flow of fluid, e.g., urine, past the locking collar 56′. If desired, the closed end 70 of the slot or channel 65 facilitates collection of any residual or inadvertently discharged fluid and protects the locking collar 56′ during use.

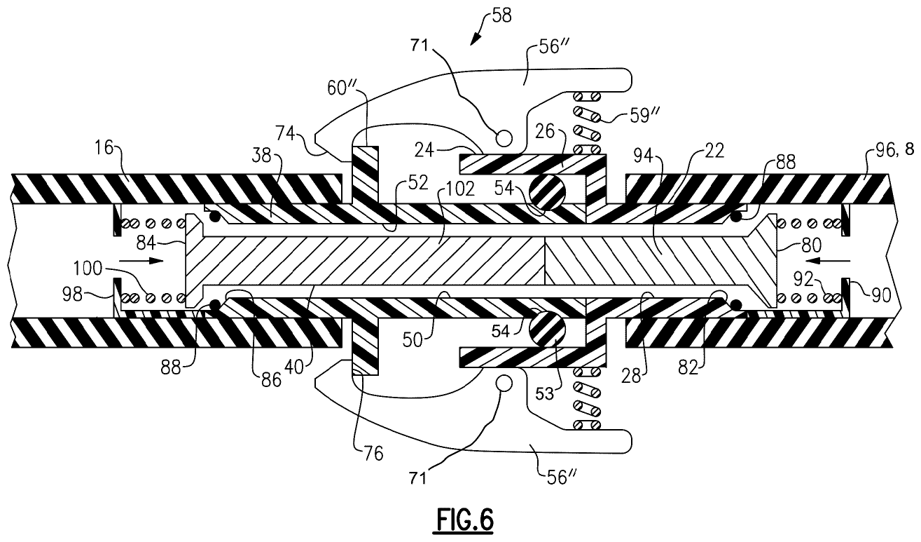

Turning now to FIG. 6, a brief description concerning the various components of a fifth embodiment of the present disclosure will now be briefly discussed. As this embodiment is similar to the first embodiment in many respects, only the differences between the fifth embodiment and the first embodiment will be discussed in detail. Similar or like elements in this embodiment are given the same reference numerals. The first important difference between the fifth embodiment and the first embodiment is the configuration of the first portion 56″ and the second portion 60″ of the mechanical quick connection mechanism 58. The second important difference is that this embodiment ensures that flow through both of the first and the second coupling devices 22, 40 is automatically interrupted immediately upon the first and the second coupling devices 22, 40 becoming separated or disconnected from one another.

According to this embodiment, the first portion of the mechanical quick connection mechanism comprises a pair of spring loaded clips 56″ which are pivotably supported by the first coupling device 22 via a pivot axle 71. It is to be appreciated that the number of spring loaded clips 56″ may be increased or decreased for some applications. A free end of each of the pair of spring loaded clips 56″ is normally biased in a direction toward one another by a respective spring 59″. The mating second portion of the mechanical quick connection mechanism comprises an annular collar 60″, which is formed integrally with the second coupling device 40. The leading end, of each one of the pair of spring loaded clips 56″, has a beveled or a chamfered surface 74 which assists with biasing the pair of spring loaded clips 56″ away from one another as the pair of spring loaded clips 56″ are brought into engagement with the annular collar 60″. As soon as the first and the second coupling devices 22, 40 are sufficiently engaged with one another, the hook face 76 of the pair of spring loaded clips 56″ are moved toward one another, by the respective springs 59″, so that the hook faces 76 of the pair of spring loaded clips 56″ engage with and retain the annular collar 60″ so as to prevent any lateral disconnecting movement of the first and the second coupling devices 20, 42 relative to one another.

When disengagement of the first and the second coupling devices 22, 40 from one another is desired, the opposite ends of the pair of spring loaded clips 56″ are biased toward one another so as to compress the springs 59″ and thereby force the hook faces 76, of the pair of spring loaded clips 56″, away from one another and the annular collar 60″, to disengage from the annular collar 60″. Once such disengagement is achieved, then the first and the second coupling devices 20, 42 can be moved laterally away from one another so as to separate and disengage the two coupling devices 22, 40 from one another.

This embodiment also includes an automatic flow interruption feature which ensures that flow through both of the first and the second coupling devices 22, 40 is automatically interrupted as soon as the first and the second coupling devices 22, 40 are separated from one another. According to this embodiment, the first cylindrical passageway 28, through the cylindrical female sleeve 26, accommodates a first sealing member 80 which is spring biased into sealing engagement with an end face 82 of the inlet of the first coupling device 22 while the second cylindrical passageway 52, through the second coupling device 40, accommodates a second sealing member 84 which is spring biased into sealing engagement with an end face 86 of the outlet of the second coupling device 40.

The first sealing member 80 is generally T-shaped and is designed to normally abut against the end face 82 of the inlet of the first coupling device 22. The end face 82 of the inlet accommodates an O-ring 88, or some other sealing element, which normally achieves a substantially fluid tight seal with the first sealing member 82. It is also possible for the O-ring or the sealing element to be carried by the first sealing member 80. A first spring cage 90 is located upstream of the first sealing member 82 and the first spring cage 90 accommodates a first sealing spring 92 which biases the first sealing member 80 into sealing engagement with the end face 82 of the inlet of the first coupling device 22 to normally close the inlet into the first cylindrical passageway 28 of the first coupling device 22. The first spring cage 90 typically has three legs (only one of which is shown in FIG. 6) which assist with equally spacing the rear wall thereof a distance away from the end face 82 so that the closing force of the first sealing spring 92 is generally along the central axis of the first cylindrical passageway 28. It is to be appreciated that the number of legs of the first spring cage 90 may be increased or decreased for some applications. The leading end of these legs each abut against the end face 82 so that the rear wall of the first spring cage 90 always remains normal to the central axis of the first cylindrical passageway 28. If desired, the leading end of these legs may be adhered or otherwise secured to the end face 82 during assembly.

The trailing end of the first sealing member 80 generally comprises an elongate first post 94 which extends within and along the first cylindrical passageway 28 of the first coupling member 22 and toward the outlet of the first coupling member 22, the purpose of which will become apparent from the following description.

A second flexible tube 96, or possible an elongated female conically shaped discharge outlet 8 of the catheter 4, surrounds and sandwiches the first spring cage 90 so as to retain the first spring cage 90 in a desired position, relative to the end face 82 of the inlet of the first coupling device 22, and prevent any lateral movement thereof away from the end face 82 of the inlet of the first coupling device 22. This arrangement ensures that the first spring cage 90 functions as a stationary surface so that the first sealing spring 92 continuously forces the first sealing member 80 toward sealing engagement with end face 82 of the inlet of the first coupling device 22.

The second sealing member 84 is generally T-shaped and is designed to abut against the end face 86 of the outlet of the second coupling device 40. The end face 86 of the outlet accommodates an O-ring 88, or some other sealing element, which normally achieves a substantially fluid tight seal with the second sealing member 84. It is also possible for the O-ring or the sealing element to be carried by the second sealing member 84. A second spring cage 98 is located downstream of the second sealing member 84 and the second spring cage 98 accommodates a second sealing spring 100 which biases the second sealing member 84 face into sealing engagement with the end face 86 of the outlet of the second coupling device 40 to normally close the outlet of the second cylindrical passageway 52 of the second coupling device 40. The second spring cage 98 typically has three legs (only one of which is shown in FIG. 6) which assist with equally spacing the rear wall thereof a distance away from the end face 86 of the outlet of the second coupling device 40 so that the closing force of the first sealing spring 92 is generally along the central axis of the second cylindrical passageway 52. It is to be appreciated that the number of legs of the second spring cage 98 may be increased or decreased for some applications. The leading end of these legs each abut against the end face 86 so that the rear wall of the second spring cage 98 always remains normal to the central axis of the second cylindrical passageway 52. If desired, the leading end of these legs may be adhered or otherwise secured to the end face 86 during assembly.

The leading end of the second sealing member 84 generally comprises an elongate second post 102 which extends within and along the second cylindrical passageway 52 of the second coupling member 40 toward the inlet of the first coupling device 22. The first flexible tube 16 surrounds and sandwiches the second spring cage 98 so as to retain the second spring cage 98 in a desired position, relative to the end face 86 of the outlet of the second coupling device 40 and prevent any lateral movement thereof away from the end face 86 of the outlet of the second coupling device 40. This arrangement ensures that the second spring cage 98 functions as a stationary surface so that the force of the second sealing spring 100 continuously forces the second sealing member 84 toward sealing engagement with the end face 86 of the outlet of the second coupling device 40.

The combined length of the first post 94 and the second post 102 is selected such that when the first and the second coupling devices 22, 40 are brought into locking engagement with one another by engagement of the pair of spring loaded clips 56″ with the locking collar 60″ (see FIG. 6), both of the first and the second sealing members 80, 84 are biased, by the first and the second posts 94, 102 against the force of the springs 92, 100, out of sealing engagement with the respective end faces 82, 86 of the inlet and the outlet of the first and the second coupling devices 22, 40 so as to permit the flow of fluid past and around the first and the second sealing members 80, 84. As a result, fluid, e.g., urine, is now permitted to flow from the catheter 4 around and past both the first spring cage 90 and the first sealing member 80, through the first and the second passageways 28, 52 along the exterior surface of and/or along any longitudinal grooves or channels formed in the exterior surface of the posts 94, 102 or the interior surfaces of the passageways 28, 52, and past both the second sealing member 84 and the second spring cage 98 into and along the first flexible tubing 16 to the collection device 18. Such flow will continue as long as the first and the second coupling devices 22, 40 remain in engagement with one another.

As soon as the first and the second coupling devices 22, 40 become disengaged from one another, the posts 94, 102 began to separate and disengage from one another and thus no longer bias the respective first and second sealing members 80, 84 out of sealing engagement with the respective end faces 82, 86 of the first and the second coupling devices 22, 40. Once this occurs, the springs 92, 100 respectively bias the first and the second sealing members 80, 84 back into sealing engagement with the end faces 82, 86 of the coupling devices 22, 40 to thereby prevent the flow of further fluid, e.g., urine, through the respective first and the second passageways 28, 52.

Since the posts 94, 102 occupy a significant amount of the space or volume contained within the first and the second passageways 28, 52, very little fluid, e.g., urine, is contained within the first and the second passageways 28, 52 upon disconnecting the first and the second coupling devices 22, 40 from one another. Moreover, any fluid remaining within the passageways 28, 52 can be easily collected or absorbed by a napkin, a towel, a cloth, a wipe or a receptacle, for example. As a result of such arrangement, the flow of fluid through both the first and the second coupling devices 20, 42 is automatically interrupted as soon as the first and the second coupling devices 20, 42 become disconnected from one another.

While the first and the second coupling devices 22, 40 are described as being either a locking collar 56 and a mating annular recess or groove 60 or a pair of spring loaded clips 56″ and an annular collar 60″, it is to be appreciated that there are a number of other mechanical quick connection mechanisms that may be utilized for selectively preventing lateral movement of the first and second coupling devices 22, 40 relative to one another to retain engagement of the first and the second coupling devices 22, 40 or permitting lateral movement of the first and second coupling devices 22, 40 relative to one another to engage or disengage the coupling devices 22, 40, without departing from the spirit and scope of the present disclosure.

While various embodiments of the present invention have been described in detail, it is apparent that various modifications and alterations of those embodiments will occur to and be readily apparent to those skilled in the art. However, it is to be expressly understood that such modifications and alterations are within the scope and spirit of the present disclosure, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense.

1. An improved catheter system comprising:
a catheter having a discharge outlet being connected to an inlet of a first coupling device;
a second end of a first flexible tube being connected to a collection device while a first end of the first flexible tube being connected to an outlet of a second coupling device;
one of the first and the second coupling devices comprising a male cylindrical sleeve and the other of the first and the second coupling devices comprising a female cylindrical sleeve, and the female cylindrical sleeve being sized to receive and form a fluid tight seal with the male cylindrical sleeve; and
the first coupling device carrying a first portion of a connection mechanism while the second coupling device carrying a mating second portion of the connection mechanism, and the connection mechanism facilitates connecting and disconnecting of the first and the second coupling devices from one another by simultaneous disconnecting the first and the second portions of the connection mechanism and sliding the first and the second coupling devices relative to and out of engagement with one another.

2. The improved catheter system according to claim 1, wherein the mating first and second portions of the connection mechanism generate a tactile sound once the female cylindrical sleeve forms the fluid tight seal with the male cylindrical sleeve.

3. The improved catheter system according to claim 1, wherein at least one O-ring is provided between the male cylindrical sleeve and the female cylindrical sleeve to form the fluid tight seal therebetween.

4. The improved catheter system according to claim 3, wherein an annular recess is formed within a surface of one of the male cylindrical sleeve and the female cylindrical sleeve, and the O-ring is retained within the annular recess to form a fluid tight seal therebetween.

5. The improved catheter system according to claim 1, wherein a first rotatable valve is located between the catheter and an outlet of the first coupling device, and when the first rotatable valve is in its closed position, fluid is prevented from flowing out of the catheter, but when the first rotatable valve is in its open position, fluid is permitted to flow out of the catheter toward the collection device.

6. The improved catheter system according to claim 5, wherein of a second rotatable valve is located between an outlet of the second coupling device and the collection device, and when the second rotatable valve is in its closed position, the flow of fluid past the second rotatable valve is prevented, but when the second rotatable valve is in its open position, the flow of fluid past the second rotatable valve toward the collection device is permitted.

7. The improved catheter system according to claim 1, wherein the collection device is one of a leg bag, a thigh bag and a waist bag, and the collection device includes a strap for securing the collection device.

8. The improved catheter system according to claim 1, wherein when the mating first and second portions of the connection mechanism engage with one another, a positive signal or indicator is generated so that a patient or service personnel knows that the mating first and second coupling devices are connected with one another.

9. The improved catheter system according to claim 1, wherein the discharge outlet of the catheter is permanently connected to the first coupling device by one of melting, welding, gluing, an adhesive, shrink fitting, and bonding, and the first flexible tube is permanently connected to the second coupling device by one of melting, welding, gluing, an adhesive, shrink fitting, and bonding.

10. The improved catheter system according to claim 1, wherein the connection mechanism comprises one of a locking collar and a mating annular recess.

11. The improved catheter system according to claim 1, wherein a spring normally biases the locking collar into a closed sealing position in which a surface of the locking collar seals the outlet of a passageway of the first coupling device so as to prevent the flow of fluid through the first coupling device.

12. The improved catheter system according to claim 11, wherein a sealing member is located between the surface of the locking collar and the outlet of the first coupling device to form a fluid tight seal therebetween.

13. The improved catheter system according to claim 11, wherein the locking collar is accommodated within a channel, and one end of the channel is a closed end which assists with collecting any residual or inadvertently discharged fluid and protects the locking collar during use.

14. The improved catheter system according to claim 1, wherein the connection mechanism comprises a pair of spring loaded clips and an annular collar, and hook faces of the pair of spring loaded clips engage with the annular collar to prevent disengagement of the first and the second coupling devices from one another.

15. The improved catheter system according to claim 1, wherein the improved catheter system has an automatic flow interruption feature which ensures that flow through both of the first and the second coupling devices is automatically interrupted when the first and the second coupling devices are separated from one another.

16. The improved catheter system according to claim 15, wherein the automatic flow interruption feature comprises a first sealing member which is normally spring biased into sealing engagement with an end face of an inlet of the first coupling device and a second sealing member which is normally spring biased into sealing engagement with an end face of an outlet of the second coupling device.

17. The improved catheter system according to claim 16, wherein a trailing end of the first sealing member generally comprises an elongate first post which extends within and along the first cylindrical passageway of the first coupling member toward an outlet of the first coupling member, and a leading end of the second sealing member generally comprises an elongate second post which extends within and along the second cylindrical passageway of the second coupling member toward the inlet of the first coupling device, and the first and the second posts force the first sealing member out of sealing engagement with the end face of the inlet of the first coupling device and the second sealing member out of sealing engagement with the end face of the outlet of the second coupling device to permit the flow of fluid past and around the first and the second sealing members.

18. The improved catheter system according to claim 17, wherein the first sealing member is generally T-shaped and is designed to normally abut against the end face of the inlet of the first coupling device and the second sealing member is generally T-shaped and is designed to abut against the end face of the outlet of the second coupling device.

19. An improved catheter system comprising:
a catheter having a discharge outlet being permanently connected to a first coupling device;
a collection device having a second end of a first flexible tube being connected to an inlet of the collection device while a first end of the first flexible tube being permanently connected to an outlet of a second coupling device;
one of the first and the second coupling devices comprising a male cylindrical sleeve and the other of the first and the second coupling devices comprising a female cylindrical sleeve, the female cylindrical sleeve being sized to receive and form a fluid tight seal with and around the male cylindrical sleeve, and one of the male and the female cylindrical sleeve supporting an O-ring for forming the fluid tight seal therebetween;
the first coupling device carrying a first portion of a connection mechanism while the second coupling device carrying a mating second portion of the connection mechanism, and the connection mechanism facilitating releasable connection and disconnection of the first and the second coupling devices from one another by simultaneously disconnecting the first and the second portions of the connecting mechanism and sliding the first and the second coupling devices away from one another and out of engagement with one another; and
when the first and the second portions of the connection mechanism are disconnected from one another, a valve of the first coupling device automatically interrupts the flow of urine out through the discharge outlet and past the first coupling device.

20. A method of providing an improved catheter system, the method comprising:
permanently connecting a first coupling device to a discharge outlet of a catheter;
permanently connecting a second end of a first flexible tube to an inlet of a collection device and permanently connecting a first end of the first flexible tube to an outlet of a second coupling device;
providing one of the first and the second coupling devices with a male cylindrical sleeve and providing the other of the first and the second coupling devices with a female cylindrical sleeve, sizing the female cylindrical sleeve to receive and form a fluid tight seal with and around the male cylindrical sleeve, and supporting an O-ring on one of the male and the female cylindrical sleeves for forming the fluid tight seal therebetween;
carrying a first portion of a connection mechanism on the first coupling device and carrying a mating second portion of the connection mechanism on the second coupling device, and facilitating releasable connection and disconnection of the first and the second coupling devices from one another, via the connection mechanism, by simultaneously disconnecting the first and the second portions of the connecting mechanism from one another and sliding the first and the second coupling devices away from one another and out of engagement with one another; and
automatically interrupting, via a pair of movable valves, the flow of fluid through the first and the second coupling devices when the first and the second portions of the connection mechanism are disconnected from one another.
